(19) United States
(12) Patent Application Publication
(10) Pub. No.: US 2015/0197774 A1
(43) Pub. Date: Jul. 16, 2015

(54) PROCESS FOR THE PRODUCTION OF ETHANOL

(71) Applicant: Estibio ApS, Ballerup (DK)

(72) Inventors: Rasmus Lund Andersen, Lyngby (DK); Karen Møller Jensen, Odense SO (DK); Marie Just Mikkelsen, Bronshoj (DK)

(21) Appl. No.: 14/424,341

(22) PCT Filed: Aug. 30, 2013
(86) PCT No.: PCT/EP2013/067984

§ 371 Date: Feb. 26, 2015

(30) Foreign Application Priority Data
Aug. 31, 2012 (GB) 1215505.7

(51) Int. Cl. C12P 7/14

(52) U.S. Cl. CPC C12P 7/14

(57) ABSTRACT

Lignocellulosic biomass is pre-treated to provide crude monosaccharides and crude polysaccharides, which are then hydrolysed in the presence of at least one enzyme to provide crude monosaccharides. These are continuously provided in an aqueous fermentation broth at a concentration such as 100 g/L along with associated inhibitory factors to a fermentation vessel containing suspended thermophilic microorganisms, and then continuously fermented at elevated temperature by said microorganisms to form ethanol. At least a portion of said ethanol is continually removed from the fermentation broth to permit the fermentation to continue despite the introduction of the inhibitory factors.

The present invention relates to a continuous method for the production of ethanol.

World ethanol production in 2011 is estimated at more than 22,300 million gallons and is rapidly increasing (Renewable fuels association, 2012 Ethanol Industry Outlook). The production of ethanol can be either from starch or sugar, which primarily consist of glucose, or from lignocellulosic material such as wood, straw, grass, or agricultural and household waste products. The main constituents of lignocellulosic material are the polymers cellulose and hemicellulose. While cellulose is a rather homogenous polymer of glucose, hemicellulose is a much more complex structure of different pentoses and hexoses. The complex composition of hemicellulose requires different means of pre-treatment of the biomass to release the sugars and also different fermenting organisms. To produce ethanol by fermentation, a microorganism able rapidly to convert sugars into ethanol with very high yields is required.

The major fermentable sugars derived from hydrolysis of various lignocellulosic materials are glucose and xylose. Microorganisms currently used for industrial ethanol production from starch materials, Saccharomyces cerevisiae and Zymomonas mobilis, are unable naturally to metabolize xylose and other pentose sugars. Considerable effort has been made in the last 20 years in the development of recombinant hexose/pentose-fermenting microorganisms for fuel ethanol production from lignocellulose sugars, however, a common problem with genetically engineered ethanologens is the so-called “glucose repression” i.e. sequential sugar utilization.

Xylose conversion starts only after glucose depletion, resulting in “xylose sparing” i.e. incomplete xylose fermentation. Achieving co-fermentation of glucose and xylose is therefore an important step in reducing ethanol production cost from lignocellulosic raw materials. Thermophilic anaerobic bacteria have the unique trait of being able to ferment the whole diversity of monomeric sugars present in lignocellulosic hydrolysates. In addition, the industrial use of thermophilic microorganisms for fuel ethanol production offers many potential advantages including high bioconversion rates, low risk of contamination, cost savings via mixing, cooling and facilitated product recovery.

These microorganisms are, however, sensitive to high sugar and ethanol concentrations and produce ethanol at low yields at high substrate concentrations. In addition, the thermophilic microorganisms are sensitive to inhibitory compounds in the lignocellulosic hydrolysates when grown in batch and cell lysis has been observed at high cell densities making it difficult to obtain the high process efficiencies required by the industry (Hemme et al., 2011).

Lignocellulosic material is the most abundant source of carbohydrate on earth. If production of ethanol from lignocellulosic biomass is to be economically favourable, all sugars including pentoses must be used. However, lignocellulosic biomass contains inhibitors that will normally be toxic to the fermenting organism. Such inhibitors include furan derivatives (furfural and 5-hydromethylfurfural (HMF)), organic acids (acetic acid, formic acid, and ferulic acid) and lignin derivatives (vanillin, 4-hydroxybenzaldehyde, guaiacol, and phenol). The toxic effect is enhanced by high levels of ethanol. Before fermentation, therefore, poly- and monosaccharides derived from lignocellulosic biomass require purification to remove such substances (see e.g. Zhang et al. Biotechnology for Biofuels, 2010, 3: 26).

The method of fermentation greatly influences efficiency and thereby cost. Fermentation in continuous systems offers several advantages over batch fermentation. The growth rate is controlled and the cells are well maintained, since fresh medium replaces the old culture while dilution takes place. High productivity per unit volume is achieved, the process is less labour intensive and less downtime is needed (Najafpour, 2007). Continuous fermentations are however difficult for most types of organisms, including yeasts, due to the risk of contamination.

Long term continuous fermentation has been demonstrated for thermophilic bacteria such as Thermoanaerobacter sp. (WO2007/134607). However, these fermentations were performed in immobilized fermentation systems, which are difficult to scale up compared to more traditional systems such as suspended cell systems, i.e. in continuous stirred tank reactors (CSTR). The immobilization matrix can prevent heat transfer and homogeneous distribution of nutrients and products, factors that will lead to lower overall ethanol yield and productivity in the reactor. A problem of using CSTR in fermentation of lignocellulose hydrolysate is that the inhibition caused by the presence of inhibitory compounds will lead to low growth rate which again leads to low productivity and risk of cell wash-out. Efficient continuous fermentation in a fully suspended system such as a CSTR has never been demonstrated for thermophilic bacteria growing on high concentrations of lignocellulosic hydrolysates without use of cell recycle systems. As we demonstrate below, attempting to run a similar fermentation using Thermoanaerobacter sp without immobilisation and without use of the present invention does indeed lead to these problems of low growth rate and cell washout.

It has been contemplated that the high temperature of thermophilic fermentation could facilitate downstream recovery of ethanol by applying a slight vacuum or using membrane technology (Taylor, 2009). It is however not disclosed there that the removal of ethanol directly from the fermentor could relieve the inhibition from other substances present in lignocellulosic hydrolysates and thereby enable fermentation of high concentrations of such hydrolysates in a suspended culture rather than immobilised system.

The use of thermophilic micro-organisms for fermentation of hydrolysed lignocellulosic material and the use of continuous fermentation are generally mentioned in WO01/60752. However, there is no exemplified demonstration of this. Ethanol removal was not used.

Batch fermentation of lignocellulosic material using a thermophilic organism is disclosed in WO2007/130984. Ethanol removal is not disclosed.

WO2010/076797 discloses fermentation of lignocellulosic hydrolysates at high dry-matter content using an inhibitor-tolerant thermophilic bacterium in a continuous fermentation in a fluidised bed reactor, rather than in a suspension culture. Ethanol removal is not used.

WO2010/151832 discloses in general terms the production of C3-C6 alcohols, but not ethanol, using thermophilic bacteria with removal of product alcohol from the fermentation. Whilst the possibility of using a lignocellulosic feedstock is mentioned, there is no demonstration of continuous fermentation of such material using thermophiles in suspension culture with alcohol removal.

WO2010/010116 discloses thermophilic fermentation of relatively low lignocellulose derived xylose concentrations of up to 12.8 g/L in an immobilized cell continuous upflow system with high ethanol yield. Ethanol removal is not disclosed.

WO2011/163373 discloses gas stripping and ethanol removal from a fermentation of glycerol using a heat tolerant micro-organism. A further disclosure of this kind is seen in US2005/0089979.

US2011/0020890 (Javed) discloses a continuous thermophilic fermentation producing ethanol using a hydrolysed biomass feedstock with ethanol removal. However, this is not demonstrated by example. It is not disclosed that in such a continuous culture the micro-organism may be in free suspension rather than immobilised where the feedstock carbohydrate or dry matter solids concentration is high and inhibitor compounds have not been removed. Javed also suggests adding high concentrations of glycerol to improve ethanol yield.

Continuous thermophilic fermentation with ethanol removal using a high sugar concentration feed is disclosed in U.S. Pat. No. 5,182,199 (Hartley). Bacillus stearothermophilus is used for the fermentation. This is facultatively anaerobic and in the Hartley process both anaerobic and aerobic fermentation steps are used. The aerobic fermentation is needed to allow the bacteria to multiply before being returned to the anaerobic fermentation.

Amartey et al (1999) show that it is necessary to recycle Bacillus stearothermophilus cells in order to ferment non-detoxified lignoellulosic hydrolysates. It is therefore one object of the present invention to provide a fermentation system and method for production of ethanol which is capable of overcoming the above-mentioned obstacles.

### Definitions

The generic term “monosaccharide” (as opposed to polysaccharide) denotes a single unit, without glycosidic connection to other such units. It includes aldoses, dialdoses, aldoketoses, ketoses and diketoses, as well as deoxy sugars and amino sugars, and their derivatives, provided that the parent compound has a (potential) carbonyl group. The term “sugar” is frequently applied to monosaccharides and lower oligosaccharides. Typical examples are glucose, fructose, xylose, arabinose, galactose and mannose.

“Polysaccharide” is the name given to a macromolecule consisting of a large number of monosaccharide residues joined to each other by glycosidic linkages. Typical polysaccharides are selected from starch, glucan, lignocellulose, cellulose, hemicellulose, glycogen, xylan, glucuronoxylan, arabinoxylan, arabinogalactan, glucomannan, xyloglucan, and galactomannan.

“Crude polysaccharides” refer to polysaccharides such as cellulose or hemicelluloses which have not been purified or refined, i.e. they are present in a mixture with other lignocellulosic biomass components such as acetic acid or lignin degradation products. Likewise, crude monosaccharides refer to monosaccharides such as glucose or xylose present in a mixture containing also other lignocellulosic biomass components.

“Continuous fermentation” is used to describe fermentations in which new monosaccharide or polysaccharide containing influent continuously replaces the fermentation broth in the reactor to allow continuous ethanol and cell production in the reactor. A continuous fermentation typically has a duration of more than two weeks.

“Thermophilic” is used to describe microorganisms that grow optimally at temperatures between 50° C. and 80° C.

“Fresh inoculum” is used to describe a volume of microorganisms that has not previously been present in a main fermentation vessel.

“Fermentable carbohydrate” is used to describe the aggregate of monosaccharides, oligosaccharides and poly saccharides fermentable by thermophilic microorganisms and determinable by the standard laboratory procedures described hereafter.

The applicant has now demonstrated that if ethanol removal is applied to the fermentation methods of WO2007/134607, it is possible to successfully avoid immobilisation of the bacteria and to use freely suspended micro-organisms even though a high concentration of carbohydrate containing feed bringing with it high levels of inhibitor compounds is used.

In a first aspect the present invention relates to a method for the production of ethanol comprising feeding a fermentable lignocellulosic biomass feed into a continuous fermentation, said fermentable lignocellulosic biomass feed having being obtained by treatment of a starting lignocellulosic biomass to liberate carbohydrates contained therein and containing the carbohydrates and associated fermentation inhibiting biomass components including at least one of hydroxymethylfurfural, 2-furaldehyde and acetic acid produced from said starting biomass together with the carbohydrates in said treatment, and continuously fermenting fermentable carbohydrate components of said biomass feed at an elevated temperature using an obligitorily anaerobic thermophilic microorganism which is suspended and not immobilised, wherein ethanol is continuously or continually removed during the fermentation and wherein said feed contains a concentration of the fermentation inhibiting compound hydroxymethylfurfural of at least 0.05 g/L, or contains a concentration of the fermentation inhibiting compound 2-furaldehyde of at least 0.5 g/L, or a concentration of the fermentation inhibiting compound acetic acid of at least 5 g/L.

The method may further comprise a preceding step of conducting said treatment of said starting lignocellulosic biomass to provide said fermentable lignocellulosic biomass feed.

Optionally, said feed contains at least two of: a concentration of the fermentation inhibiting compound hydroxymethylfurfural of at least 0.05 g/L, a concentration of the fermentation inhibiting compound 2-furaldehyde of at least 0.5 g/L, and a concentration of the fermentation inhibiting compound acetic acid of at least 5 g/L.

Alternatively, said feed contains all of: a concentration of the fermentation inhibiting compound hydroxymethylfurfural of at least 0.05 g/L, a concentration of the fermentation inhibiting compound 2-furaldehyde of at least 0.5 g/L, and a concentration of the fermentation inhibiting compound acetic acid of at least 5 g/L.

In an alternative aspect, the invention provides a method for the production of ethanol comprising feeding a fermentable lignocellulosic biomass feed into a continuous fermentation, said fermentable lignocellulosic biomass feed having being obtained by treatment of a starting lignocellulosic biomass to liberate carbohydrates contained therein and containing the carbohydrates and associated fermentation inhibiting biomass components including 2-furaldehyde and acetic acid produced in said treatment from said starting biomass together with the carbohydrates, and continuously fermenting fermentable carbohydrate components of said biomass feed at an elevated temperature such as at least 50° C. using an obligatorily anaerobic thermophilic microorganism which is suspended and not immobilised, wherein ethanol is continuously or continually removed during the fermentation and wherein said feed has a concentration of the fermentation inhibiting compound 2-furaldehyde of at least 0.5 g/150g of fermentable carbohydrate, and/or a concentration of acetic acid of at least 5 g/150g of fermentable carbohydrate, and/or a concentration of hydroxymethylfurfural of at least 0.05 g/150 g of fermentable carbohydrate. When the fermentation is of essentially C5 sugars from hydrolysis of hemicellulose without the C6 sugars from cellulose hydrolysis, the amount of the inhibitors present may be such that the feed has a concentration of the fermentation inhibiting compound 2-furaldehyde of at least 0.5 g/60 or per 70 g of fermentable carbohydrate, and/or a concentration of acetic acid of at least 5 g/60 or per 70 g of fermentable carbohydrate, and/or a concentration of hydroxymethylfurfural of at least 0.05 g/60 or per 70 g of fermentable carbohydrate. Where both C5 and C6 sugars are to be fermented, the ratio of inhibitor to carbohydrate expected will be less.

The inhibitor concentrations may for instance be such that the feed has a concentration of the fermentation inhibiting compound 2-furaldehyde of at least 0.5 g/100 g of fermentable carbohydrate, and/or a concentration of acetic acid of at least 5 g/100 g of fermentable carbohydrate, and/or a concentration of hydroxymethylfurfural of at least 0.05 g/100 g of fermentable carbohydrate.

The fermentable carbohydrate content of the feed includes sugar monomers, dimers, oligomers, cellulose and hemicellulose and these can be measured using standard procedures i.e. from the National Renewable Energy Laboratories (‘Determination of Sugars, Byproducts, and Degradation Products in Liquid Fraction Process Samples’ Laboratory Analytical Procedure (LAP) Issue Date: Dec. 8, 2006 A. Sluiter, B. Hames, R. Ruiz, C. Scarlata, J. Sluiter, and D. Templeton; ‘Determination of Structural Carbohydrates and Lignin in Biomass’ Laboratory Analytical Procedure (LAP)Issue Date: April 2008 Revision

Date: July 2011 (Version 07-08-2011) A. Sluiter, B. Hames, R. Ruiz, C. Scarlata, J. Sluiter, D. Templeton, and D. Crocker; ‘Preparation of Samples for Compositional Analysis’ Laboratory Analytical Procedure (LAP) Issue Date: Aug. 6, 2008 B. Hames, R. Ruiz, C. Scarlata, A. Sluiter, J. Sluiter, and D. Templeton, ‘Determination of Insoluble Solids in Pretreated Biomass Material’ Laboratory Analytical Procedure (LAP) Issue Date: Mar. 21, 2008 A. Sluiter, D. Hyman, C. Payne, and J. Wolfe.

The concentration of 2-furaldehyde, alternatively known as furan-2-carbaldehyde, furfural, furan-2-carboxaldehyde, fural, furfuraldehyde, or pyromucic aldehyde, may be measured by HPLC or GC, as may the concentration of acetic acid.

Methods of the invention may further comprise a preceding step of conducting said treatment of said starting lignocellulosic biomass to provide said fermentable lignocellulosic biomass feed. Such a treatment may include pre-treating said lignocellulosic biomass to liberate crude C5 monosaccharides and to liberate crude polysaccharides for hydrolysis. This pre-treatment may be followed by hydrolysing said polysaccharides to provide crude C6 monosaccharides in said feed. Such hydrolysis of said crude polysaccharides may be conducted by enzymes added to the pre-treated lignocellulosic biomass. Alternatively, hydrolysis of said polysaccharides may be conducted by enzymes produced in or added to said fermentation, or these methods may be combined.

Ethanol may be removed from the fermentation by gas stripping using a stripping gas. This will generally be oxygen free. Ethanol may be removed from admixture with the stripping gas and the thus purified stripping gas may be reused for ethanol removal. Alternatively, ethanol may be removed from the fermentation by the use of vacuum.

Optionally, said removal of ethanol is conducted on a liquid stream withdrawn from the fermentation into a separate vessel from a vessel in which said fermentation is carried out.

The microorganism may be a filamentous microorganism.

It may be from the class of Clostridia. The microorganism may be from the order of Thermoanaerobacteriales. The microorganism may be from the family of Thermoanaerobacteriaceae. The microorganism may be from the genus of Thermoanaerobacter.

The microorganism is preferably selected from the group consisting of Thermoanaerobacter acetoethylicus, Thermoanaerobacter brockii, Thermoanaerobacter ethanolicus, Thermoanaerobacter inferii, Thermoanaerobacter italicus, Thermoanaerobacter italicus subsp. marato, Thermoanaerobacter keratinophilus, Thermoanaerobacter kivui, Thermoanaerobacter mathranii, Thermoanaerobacter pseudethanolicus, Thermoanaerobacter siderophiles, Thermoanaerobacter sulfurigignens, Thermoanaerobacter sulfurqphilus, Thermoanaerobacter thermocopriae, Thermoanaerobacter thermohydrosulfuricus, Thermoanaerobacter uzonensis and Thermoanaerobacter wiegelii.

The microorganism may be genetically modified by deletion or inactivation of genes involved in production of acetic acid, lactic acid or other by-products to increase the yield of ethanol. It may also be modified by deletion or inactivation of genes involved in sporulation. The microorganism may also have inserted genes such as genes involved in carbohydrate degradation, uptake, transport or metabolism or it may have modified activity of genes involved in maintaining the correct redox balance. The micro-organism may be one described in any of WO01/60752, WO2007/134607, WO2010/010116 and WO2011/076797. In particular, the microorganism may be one in which there has been deletion or inactivation of genetic material encoding L-lactate dehydrogenase and deletion or inactivation of genetic material encoding of acetate kinase and/or phosphotransacetylase.

The carbohydrate content of said feed is at least 100 g/L, but more preferably is at least 125 g/L, and optionally is at least 150 g/L.

Optionally, a portion of the fermentation broth is removed during the continuous fermentation process, and the microorganisms are recycled back into the fermentation vessel. This may be carried out by:
isolating a portion of the fermentation broth,
isolating microorganisms from said portion of fermentation broth,
optionally, treating said isolated microorganisms, and
re-introducing said isolated microorganisms into the fermentation broth.

To release the nutrients inside the cells into the medium thereby providing nutrients to the fermentation, the isolated microorganisms may be treated using a treatment selected from the group consisting of heat treatment, acid or base treatment and enzymatic lysis. Each of these may be done with or without increased pressure.

Isolating of the microorganisms may be performed by centrifugation, optionally continuous centrifugation. Alternatively, isolation of the microorganisms may take place via filtration.

The rate of carbohydrate feed to the fermentation is preferably at least 0.5, more preferably at least 1.0, or at least 2 or 4 or 5 g of carbohydrate per litre of fermentation volume per hour.

It is not intended that the treated lignocellulosic biomass will be further treated to remove inhibitors prior to fermentation either at all or at least to any significant extent. Accordingly, it is expected that the fermentable lignocellulosic biomass feed contains all of, or at least 80% of, the associated fermentation inhibiting biomass components produced from said starting biomass together with the carbohydrates in said treatment.

However, optionally lignin is removed from the biomass following said treatment and prior to feeding to said fermentation. Alternatively, the level of some inhibitors may be reduced by an evaporation step prior to fermentation.

Where a pre-treatment provides crude C5 monosaccharides, these may be fed to the fermentation without enzymatic hydrolysis of crude polysaccharides to provide further C6 monosaccharides. Similarly, separated crude polysaccharides may be hydrolysed to produce crude C6 monosaccharides and these may be fed to the fermentation without the C5 monosaccharides produced earlier. Preferably however, the fermentable lignocellulosic biomass feed contains both the C5 and C6 monosaccharides liberated in said treatment. Enzymatic hydrolysis may also take place partially or solely in the fermentation vessel.

Optionally, a C5 and C6 containing feed may be subjected to a C6 fermentation by, for instance, a yeast leaving residual C5 sugars that are then subjected to a fermentation according to the invention.

Preferred and illustrative specific embodiments of the invention will be described with reference to the enclosed schematic figures, in which:

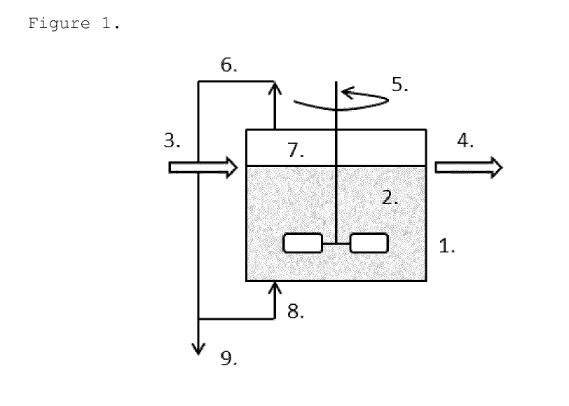

FIG. 1 is a schematic illustration of the method according to the invention.

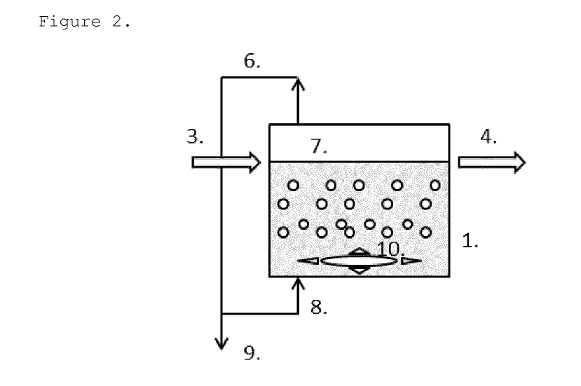

FIG. 2 is a schematic illustration of an alternative method according to the invention.

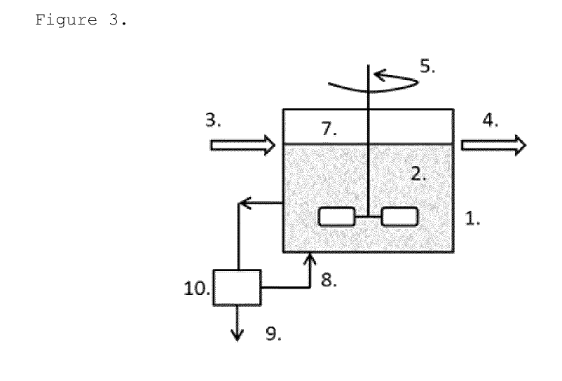

FIG. 3 is a schematic illustration of an alternative method according to the invention.

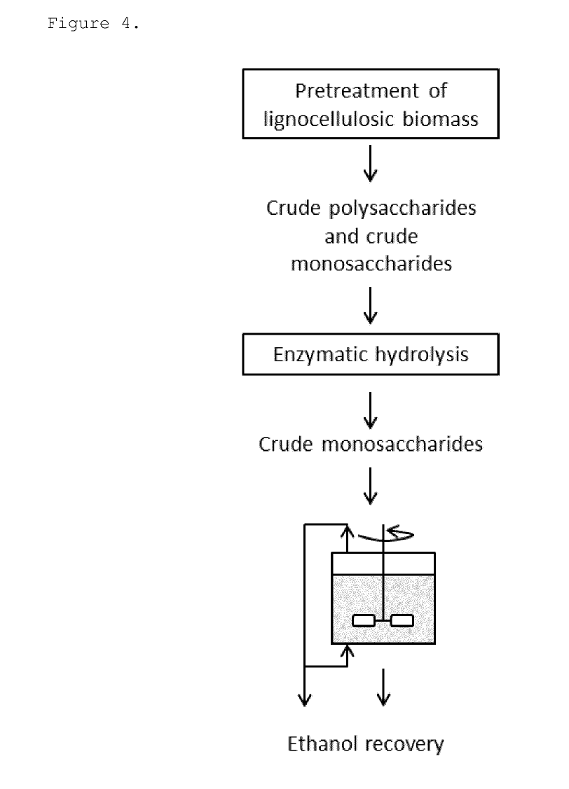

FIG. 4 is a schematic diagram of an exemplified process flowchart according to the invention in which enzymatic hydrolysis and fermentation takes place in separate vessels.

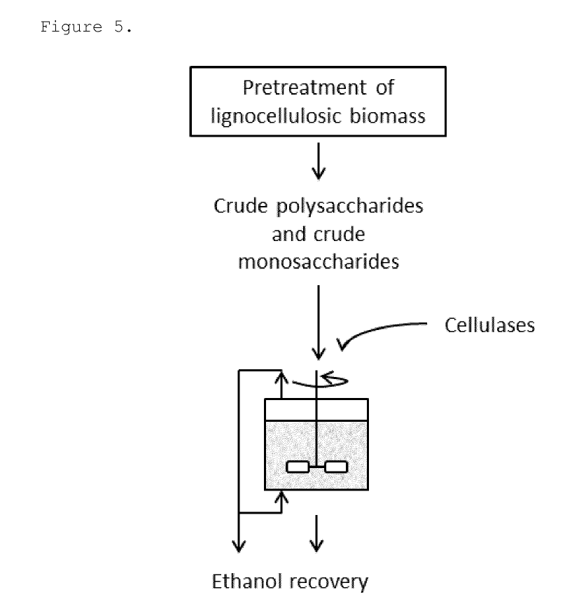

FIG. 5 is a schematic diagram of an exemplified process flow according to the invention in which enzymatic hydrolysis and fermentation takes place in the same vessel.

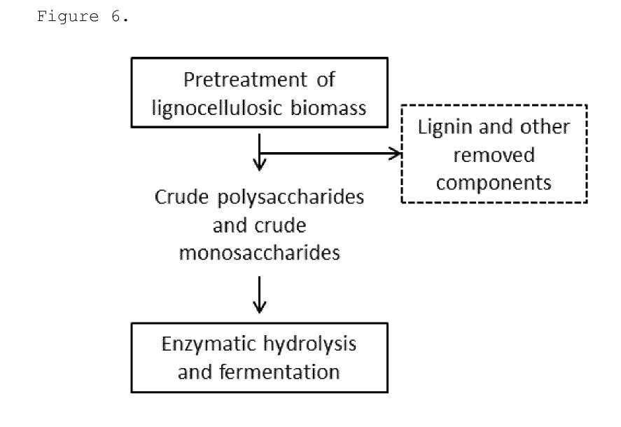

FIG. 6 shows an example in which lignin and other components are removed before enzymatic hydrolysis.

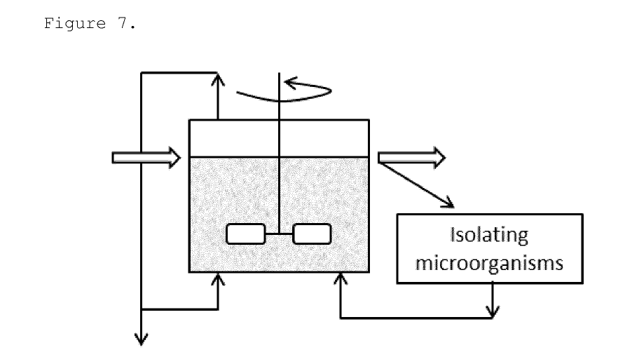

FIG. 7 is a schematic drawing of an apparatus according to the invention in which microorganisms are isolated from the fermentation broth and subsequently reintroduced into the fermentation broth.

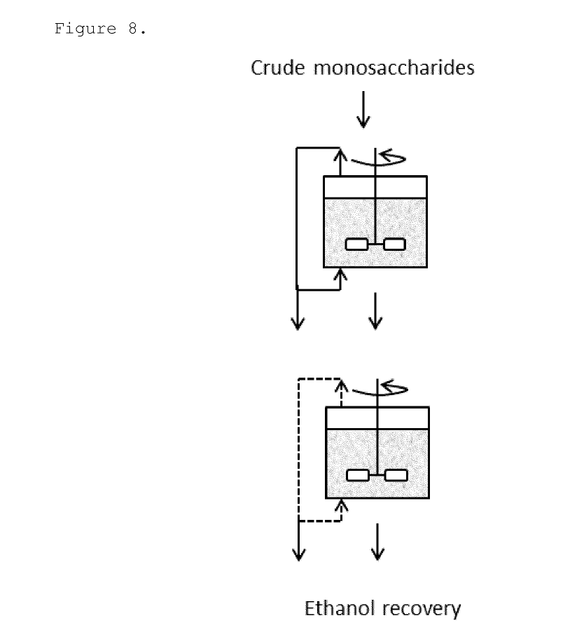

FIG. 8 is a schematic diagram of an exemplified method according to the invention in which more than one sequential fermentation vessel is employed.

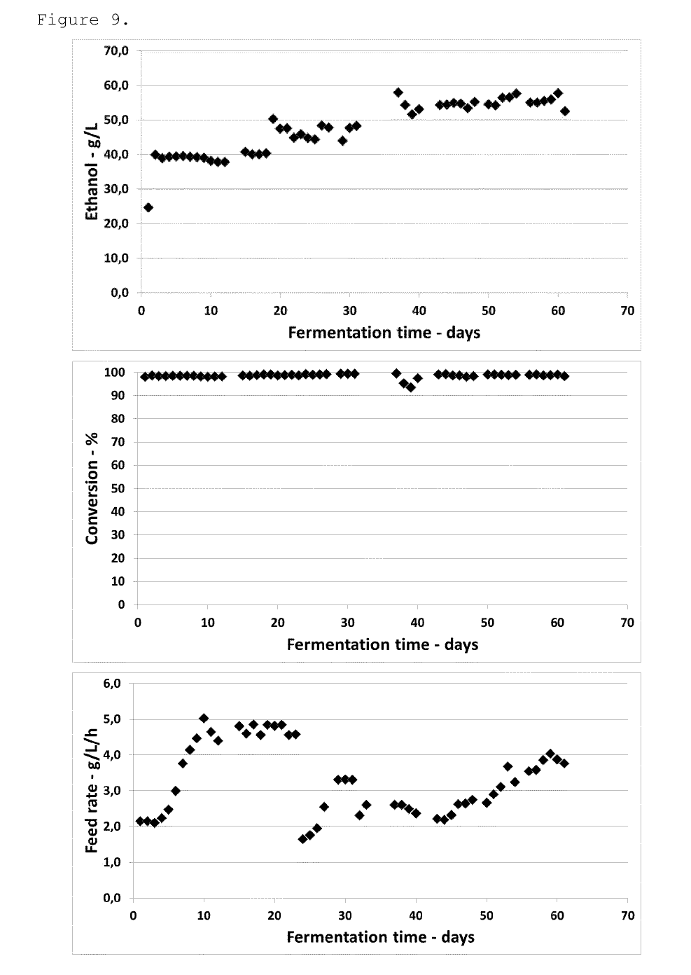

FIG. 9. Fermentation data for Example 1. The fermentation ran for more than 2 months. During this time the sugar load was increased in steps as seen by the ethanol concentration produced (upper panel) as close to 100% of the sugar was converted (middle panel). To secure the complete conversion the feed rate was adjusted on a daily basis (lower panel).

The invention provides a continuous method for the production of ethanol. The method of the invention uses lignocellulosic biomass as a starting material. Useful lignocellulosic biomass may, in accordance with the invention, be derived from plant material, such as straw, hay, garden refuse, house-hold waste, wood, fruit hulls, seed hulls, corn hulls, oat hulls, soy hulls, corn fibres, stovers, corn cobs, milkweed pods, leaves, seeds, fruit, grass, wood, paper, algae, cotton, hemp, flax, jute, ramie, kapok, bagasse, mash, distillers grains, oil palm residues, corn, sugar cane, sorghum, ensiled biomasses, Jatropha, and sugar beet.

The first step of a preferred method requires pre-treating a sample of lignocellulosic biomass to provide crude polysaccharides and crude monosaccharides which will normally be principally C5 monosaccharides. In this step, the cellulose and/or the hemicellulose in the lignocellulosic biomass material becomes more susceptible to enzymatic degradation and may be converted partially or completely into sugar monomers. Pre-treatment may be selected from acid hydrolysis, steam explosion, wet oxidation, wet explosion and enzymatic hydrolysis, or combinations thereof.

The pre-treatment method most often used is acid hydrolysis, where the lignocellulosic material is subjected to an acid such as sulphuric acid, hydrochloric acid or acetic acid and whereby the sugar polymers cellulose and hemicellulose are partly or completely hydrolysed to their constituent sugar monomers. Another type of lignocellulose hydrolysis is steam explosion, a process comprising heating of the lignocellulosic material by steam injection to a temperature of 190-230° C. A third method is wet oxidation wherein the material is treated with oxygen at 150-185° C. Other types of pre-treatment include ‘organosols’ pre-treatment using organic acids or alcohols, supercritical extraction, hot water pre-treatment, ammonia fiber explosion (AFEX), strong acid pre-treatment and lime pre-treatment.

After pre-treatment, the sugars derived from hemicelluloses or lignin may be separated from the cellulose fiber using e.g. centrifuges, filters or by precipitation.

In the second step of the illustrative method, crude polysaccharides obtained from the first step are hydrolysed, optionally in the presence of at least one enzyme, to provide crude monosaccharides. The purpose of such an additional hydrolysis treatment is to hydrolyse oligosaccharide and possibly polysaccharide species produced during the pre-treatment of cellulose and/or hemicellulose to form fermentable sugars (e.g. glucose, xylose, arabinose and possibly other monosaccharides). Such further treatments may be either chemical or enzymatic. Chemical hydrolysis is typically achieved by treatment with an acid, such as treatment with aqueous sulphuric acid, at a temperature in the range of about 100-150° C. Enzymatic hydrolysis is typically performed by treatment with one or more appropriate carbohydrase enzymes such as cellulases including endoglucanases, exoglucanases and cellobiohydrolases, glucosidases including beta-glucosidases and hemicellulases including xylanases, arabinofuranosidases, endo-xylanases and betaxylosidases at a temperature in the range of about 35-100° C.

In certain embodiments enzymes are added directly to the fermentation vessel, the so-called simultaneous saccharification (hydrolysis) and fermentation process as exemplified in FIG. 5. In other embodiments, the saccharification (hydrolysis) and fermentation take place in separate vessels as exemplified in FIG. 4.

In the third step of the illustrative method, crude monosaccharides in an aqueous fermentation broth at a concentration of at least 100 g/L along with associated inhibitory factors are continuously or continually provided to a fermentation vessel containing thermophilic microorganisms.

Suitably, the monosaccharides are present in the fermentation broth in a total concentration of at least 125 g/L, more preferably at least 150 g/L. This allows a high concentration of ethanol to be produced which will reduce the cost of ethanol recovery.

The production of inhibitors in the treatment of the feedstock will be expected to result in the fermentation broth additionally comprise at least 5 g/L acetic acid and/or at least 0.5 g/L of 2-furaldehyde, and/or at least 0.05 g/L hydroxymethylfurfural (HMF) as well as other inhibitory compounds. The concentrations of the inhibitors will probably be higher, e.g. 0.02, 0.05, 0.5, or 2.0 g/L HMF 0.5 g/L, 1 g/L or 2 g/L 2-furaldehyde and 8 g/L or 11 g/L acetic acid.

The fermentation broth may additionally comprise nitrogen, phosphorous, calcium, magnesium, manganese, cobalt, copper, boron, molybdenum, aluminium, nickel, selenium and iron salts, corn steep liquor, yeast extract, soy protein, and yeast lysate.

Useful examples of fermentation vessels include continuous stirred tank bioreactors, airlift bioreactors, bubble column bioreactors, trickle bed bioreactors, fluidized bed bioreactor. Most suitably the fermentation vessel is a continuous stirred-tank reactor. Suitably, at least one sequential fermentation vessel is employed (e.g. FIG. 7). The fluid in the vessel may be mixed using impellers, gas, liquid circulation, or combinations of these.

The aqueous fermentation broth may then be continuously fermented in the presence of the microorganisms to form ethanol. The fermentation step is suitably operated at a temperature in the range of 40-95° C., such as 50-90° C., such as 60-85° C., such as 60-70° C.

The fermentation step is suitably operated at a pH value in the range of 5.5-8, such as 6.5-7.5, such as 6.8 to 7.2.

The concentration of cells in the fermentation is suitably in the range of 2-20 g/L (dry cell weight per liter of active volume), preferably 3-15 g/L, more preferably 5-10 g/L.

Suitably less than 1 g/L/d (gram of cells per liter of fermentation volume per day), preferably less than 0.25 g/L/d, more preferably less than 0.1 g/L/d, more preferably less than 0.01 g/L/d of fresh inoculum is added to the fermentation vessel during continuous operation.

Suitably the fermentation is started as a batch fermentation and then subsequently shifted to continuous operation, such continuous operation proceeding for a period such as at least three weeks, such as at least 6 weeks, such as at least 12 weeks, such as at least 18 weeks, such as at least 24 weeks, such as at least 36 weeks.

The influent to the fermentation vessel suitably contains crude polysaccharides and crude monosaccharides corresponding to a total sugar monomer concentration of at least 50 g/L, preferably at least 100 g/L, more preferably at least 125 g/L, more preferably at least 150 g/L, and more preferably at least 200 g/L.

Preferred micro-organisms include bacteria of the genus Thermoanaerobacter and may be selected from the group consisting of Thermoanaerobacter acetoethylicus, Thermoanaerobacter brockii, Thermoanaerobacter brockii subsp. brockii, Thermoanaerobacter brockii subsp. finnii, Thermoanaerobacter brockii subsp. finnii Ako-1, Thermoanerobacter brockii subsp. Lactiethylicus, Thermoanaerobacter ethanolicus, Thermoanaerobacter ethanolicus CCSD1, Thermoanaerobacter ethanolicus JW 200, Thermoanaerobacter inferii, Thermoanaerobacter italicus, Thermoanaerobacter italicus Ab9, Thermoanaerobacter italicus subsp. marato, Thermoanaerobacter keratinophilus, Thermoanaerobacter kivui, Thermoanaerobacter mathranii, Thermoanaerobacter mathranii subsp. alimentarius, Thermoanaerobacter mathranii subsp. mathranii, Thermoanaerobacter mathranii subsp. mathranii str. A3, Thermoanaerobacter pseudethanolicus, Thermoanaerobacter pseudethanolicus ATCC 33223, Thermoanaerobacter siderophilus, Thermoanaerobacter siderophilus SR4, Thermoanaerobacter sulfurigignens, Thermoanaerobacter sulfurophilus, Thermoanaerobacter thermocopriae, Thermoanaerobacter thermohydrosulfuricus, Thermoanaerobacter uzonensis, Thermoanaerobacter wiegelii, Thermoanaerobacter wiegelii Rt8.B1, Thermoanaerobacter sp. 1004-09, Thermoanaerobacter sp. 16AFV, Thermoanaerobacter sp. 185d2, Thermoanaerobacter sp. 185g3, Thermoanaerobacter sp. 185g5, Thermoanaerobacter sp. 18AF, Thermoanaerobacter sp. 266G10, Thermoanaerobacter sp. 266y3, Thermoanaerobacter sp. 2MCR, Thermoanaerobacter sp. 3MCR, Thermoanaerobacter sp. 4MCR, Thermoanaerobacter sp. 518-21, Thermoanaerobacter sp. 711-75, Thermoanaerobacter sp. 9AFV, Thermoanaerobacter sp. A3N, Thermoanaerobacter sp. AND32, Thermoanaerobacter sp. ATCC 53627, Thermoanaerobacter sp. BKH1, Thermoanaerobacter sp. BSB-2, Thermoanaerobacter sp. BSB-21, Thermoanaerobacter sp. BSB-27, Thermoanaerobacter sp. BSB-30, Thermoanaerobacter sp. BSB-31, Thermoanaerobacter sp. BSB-33, Thermoanaerobacter sp. BSB-4, Thermoanaerobacter sp. BSB-5, Thermoanaerobacter sp. BSB-9, Thermoanaerobacter sp. EB3.8, Thermoanaerobacter sp. HA2, Thermoanaerobacter sp. HL-3, Thermoanaerobacter sp. JCM 7503, Thermoanaerobacter sp. JN 2, Thermoanaerobacter sp. K14, Thermoanaerobacter sp. K165D, Thermoanaerobacter sp. K67, Thermoanaerobacter sp. KA2, Thermoanaerobacter sp. KB4, Thermoanaerobacter sp. LD-2008, Thermoanaerobacter sp. MET-G, Thermoanaerobacter sp. NA1, Thermoanaerobacter sp. NB3, Thermoanaerobacter sp. RH0802, Thermoanaerobacter sp. RH0803, Thermoanaerobacter sp. RH0804, Thermoanaerobacter sp. RH0805, Thermoanaerobacter sp. RH0806, Thermoanaerobacter sp. RH0807, Thermoanaerobacter sp. RH0808, Thermoanaerobacter sp. SC-2, Thermoanaerobacter sp. SC-5, Thermoanaerobacter sp. SOB-1, Thermoanaerobacter sp. TC10, Thermoanaerobacter sp. TC11, Thermoanaerobacter sp. TC41, Thermoanaerobacter sp. TC44, Thermoanaerobacter sp. TC46, Thermoanaerobacter, p. TC47, Thermoanaerobacter sp. TC49, Thermoanaerobacter sp. TLO, Thermoanaerobacter sp. TPI, Thermoanaerobacter sp. W2-7_{—}661-2, Thermoanaerobacter sp. X513, Thermoanaerobacter sp. X514, Thermoanaerobacter sp. X561, Thermoanaerobacter sp. xyl-d.

The majority of the microorganisms in the fermentation vessel are suspended in the fermentation broth (i.e. they are not fastened actively or passively to a solid support).

During fermentation, at least a portion of the ethanol is removed from the fermentation broth. Suitably, ethanol is removed by gas stripping directly in the fermentation vessel and at least a part of the stripping gas is recycled into the fermentation mixture, thus reducing the amount of gas used in the overall process. Suitable stripping gases include carbon dioxide, nitrogen, or combinations of these gases. In another aspect, ethanol is removed by applying a vacuum to the fermentation vessel. In yet another aspect, the ethanol is removed from the gas phase of the fermentation by passing the gas through a system including a means for removal of ethanol such as a membrane, an adsorbent, a vacuum zone, an extractant, or a zone with increased temperature.

In another aspect, ethanol is removed from the fermentation broth by removing a portion of the fermentation broth from the fermentation vessel, partially removing ethanol from said portion, and returning the broth to the fermentation vessel thereby decreasing the concentration of ethanol in the fermentation broth. Such partial removal of ethanol is suitably achieved using vacuum distillation, membrane filtration, adsorption, pervaporation, evaporation, or distillation. Such a return of broth does not constitute or contribute to addition of fresh inoculum.

In one aspect, ethanol is removed partially. The concentration of ethanol in said fermentation medium is preferably kept below 45 g/L, more preferably below 35 g/L, optionally below 25 or 20 g/L. The proportion of the ethanol produced in the fermentation which is removed may be at least 10%, e.g. from 10-20%, particularly when the fermentation is of C5 sugars substantially only, i.e. with separation of cellulose from soluble sugars and fermentation of the soluble sugars only. Alternatively, the proportion removed may be at least 50%, e.g. from 60 to 70%, particularly where the fermentation includes or consists of fermentation of C6 sugars, i.e. including the hydrolysis products of cellulose.

Overall the proportion removed may be preferably 30-90% w/w of the ethanol production of the fermentation. The isolated ethanol may be purified, preferably via distillation.

FIGS. 1 and 2 illustrate preferred methods of the invention during continuous ethanol production. Fermentation vessel 1 contains the fermentation broth 2. The fermentation broth contains the thermophilic microorganism as well as nutrients necessary for fermentation. Influent 3 is continuously added to the fermentation vessel and effluent 4 continuously exits from the fermentation vessel. The fermentation vessel is mixed by a mixer 5 to ensure uniform distribution of heat and fermentation of broth components. To decrease the total inhibition in the fermentation broth, part of the ethanol from the fermentation is continuously removed 6 from the liquid and/or gas phase of the vessel 1 during fermentation. Ethanol 9 is recovered from the gas phase 7 by condensation, membrane filtration or absorption of ethanol, followed by recycling of the gas to the lower part of the fermentation vessel 1 via inlet 8. The influent 3 to the fermentation vessel contains crude monosaccharides derived from lignocellulosic biomass in a concentration of at least 100 g/L. It is contemplated that a vacuum can be applied to the gas phase to facilitate recovery of ethanol. Part of the ethanol produced in the fermentation vessel will exit the vessel with the fermentation effluent 4.

The method in FIG. 2 is similar to that of FIG. 1, but mixing is achieved by gas sparging via sparger 10 (bubble column bioreactor or airlift bioreactor).

The method in FIG. 3 is similar to that of FIG. 1, but the partial ethanol removal is achieved by withdrawing a liquid stream from the fermentation vessel, partially removing the ethanol from this separate stream, and returning the liquid stream with reduced ethanol content to the fermentation vessel.

Hydrolysis and fermentation steps may take place in separate reaction vessels (FIG. 4). However, in a particular embodiment, hydrolysis and fermentation steps take place together in the fermentation vessel (FIG. 5).

Combining the enzymatic hydrolysis and fermentation in one vessel may have the advantages of more efficient enzymatic hydrolysis due to continuous conversion of sugars in the broth, thereby relieving feedback inhibition (or product inhibition) on the enzymes. Other advantages of such combined fermentation system include higher product yields, decreased risk of contamination, smaller total vessel volume and simpler operation.

A purification step may take place prior to enzyme hydrolysis. This is exemplified in FIG. 6, in which lignin is removed. Some partial removal of other components such as acetic acid, levulinic acid, formic acid, lignin degradation products, or furfural may also take place.

In a particular embodiment, illustrated in FIG. 7, a portion of the fermentation broth is removed during the continuous fermentation process, and the microorganisms are recycled back into the fermentation vessel.

In particular, microorganisms may be recycled by:
a. isolating a portion of the fermentation broth,
b. isolating microorganisms from said portion of fermentation broth,
c. optionally, treating said isolated microorganisms,
d. re-introducing said isolated microorganisms into the fermentation broth

Treatment of said isolated microorganisms may be carried out by heat treatment, acid or base treatment with or without increased pressure, and enzymatic lysis.

Isolation of the microorganisms may take place via centrifugation or via microfiltration or ultrafiltration. Centrifugation techniques include disk-bowl centrifuges (Brethauer and Wyman, 2010), scroll decanters, disc-stack centrifuges, multi-chamber centrifuges, and tubular bowl centrifuges

Physical methods for treatment of said isolated microorganisms include methods for cell disruption (i) Ultrasonication, (ii) Osmotic shock (used for releasing hydrolytic enzymes and binding proteins from gram-negative bacteria), (iii) Heat Shock treatment, (iv) High pressure homogenization, (v) Impingement which involves hitting a stationary surface or a second stream of suspended particles with a stream of suspended cells at high velocity and pressure, (vi) Grinding with glass beads where the cells mixed with glass beads are subjected to a very high speed in a reaction vessel.

Chemical methods for treatment of said isolated microorganisms include treatment with alkalis, organic solvents, and detergents to lyse the cells and release the content.

Organic solvents like methanol, ethanol, isopropanol, butanol etc. can also be applied to disrupt the cells. Ionic detergents such as e.g. cationic-cetyl trimethyl ammonium bromide or anionic-sodium lauryl sulphate, can be used to denature the membrane proteins and lyse the cells. In addition, the enzyme lysozyme can be used to lyse the cells.

The fermentation may be take place in more than one fermentation vessel as illustrated in FIG. 8. The partial ethanol removal may take place in one or more fermentation vessel.

### EXAMPLES

### Materials and Methods

### Cultivation and Isolation

Pentocrobe 3120-411 (Thermoanaerobacter italicus) was originally isolated on solid surface cultivation medium using Hungate Roll Tubes (Hungate 1969) and adapted to fermentation conditions through several generations in fully suspended reactors.

### HPLC

Sugars and fermentation products were quantified by HPLC-RI using a Dionex Ulitimate 3000 (Dionex corp., USA) fitted with an Rezex ROA-organic Acid 300×7.8mm (Phenomenex, USA) combined with a SecurityGuard Cartridge Carbo-H 4*3.0 mm. The analytes were separated isocratically with filtrated (0.22 μm) 4 mM H₂SO₄ and at 60° C. Samples were centrifuged at 14.000 G for 10 minutes. All analytes were diluted to a maximum of 20 g/L using MQ-water.

### Substrates and Chemicals

Pretreated material: Hammer milled wheat straw was pre-treated in a continuous pre-treatment system (BioGasol ApS WO2010081476, WO2010081477, WO2010081478). The parameter settings were 165° C. and 0.4% v/v sulphuric acid with a retention time 15 minutes. In Example 1, the resulting material was diluted to 20% DM and separated on a Larox filter (Uototec, Finland). The liquid fraction (C5 liquor) contained (g/L): glucose, 6.2; xylose, 46.8 and arabinose, 4.8. In Example 2 the pretreated material was pH-adjusted to 5.0 using 10M NaOH and enzymatically hydrolyzed. The material was separated centrifugally in an Allegra 25R centrifuge (Beckman Coulter, USA). The substrate concentrations were (g/L): glucose, 71.8; xylose, 56.9 and arabinose, 7.5. No removal of soluble fermentation inhibitors was carried out.

### Fermentation Setup & Strategy

All fermentations were carried out using suspended cells in water jacketed glass reactors with working volumes of 575 ml and a height/width ratio of around 6:1. Stirring was introduced by a magnetic bar of 6×25 mm and an IKA-Digital stirrer (Germany) operating at 350 rpm. Hot water was recirculated from a GD120 water bath (Grant, England) and fed parallel to either two or three identical reactors.

The sparging gases used were of high purity (4.5 e.g. 99.995%) and were pressure regulated by reduction valves (Lab Line DL230 N₂ and CO₂ from Strandmøllen, Denmark) to around 0.5 bar. The actual gas flow was followed and regulated by rotometers mounted on Applikon equipment (ADI1025/ADI1010, The Netherlands). A needle-perforated sparger, made from pressure tubing, was fitted in the reactor and the used gas flow varied between 0.2 and 0.5 VVM (volume per volume per minute), depending on the HPLC determined ethanol concentrations, maintaining a reactor concentration below 25 g/L.

The entire reactor system was autoclaved at 121° C. for 30 minutes, filled with sterile basal anaerobic medium (BA) (Larsen et al. 1997) supplemented with 2 g/L yeast extract and inoculated with a fresh culture of Pentocrobe 3120-411. Liquid samples for HPLC were taken from a sampling port located at the reactor top. The pH was maintained at 7.0 by addition of NaOH (2 M).

All media were prepared following a standard procedure and sterility was obtained by autoclaving. Carbon- and nitrogen sources were handled separately preventing undesired Maillard reactions during the sterilization process. Antifoam 204 (Sigma Aldrich, Germany) was added in concentrations ranging between 0.1 and 0.2%.

### Example 1

### Continuous Thermophilic Fermentation on Laboratory Scale Using CO₂ Stripping

A continuous reactor system was set up as described in materials and methods. All influents were prepared using C5 liquor produced as above to which for some fermentations was added dextrose monohydrate (Roquette, France) in a concentration simulating the result of an enzymatic hydrolysis with 80% efficiency. Batch conditions were maintained for 18 hours before the continuous wheat straw based influent was started using a hydraulic retention time of around 30 hours. CO₂ sparging was initiated after approximately two days of fermentation, as the ethanol concentration within the reactor reached 12 g/L. The maximum ethanol removal rate was around 2.4 g/L/h. Inhibitors (e.g. HMF and 2-furaldehyde) were undetectable in the effluent, indicating that they were metabolized. The hydraulic retention time was gradually decreased until ethanol productivities exceeded 2 g/L/h.

The highest fermented influent sugar concentration in this Example was 123 g/L (total concentration) and the recovered ethanol concentration at this influent concentration was 55 g/L (6.9% vol/vol). Carbohydrate feed concentrations are shown in Table 1 (A) below.

Increasing ethanol titers (in condensed effluents) were tested throughout the fermentation by decreasing the amount of water added during feed preparation, thus introducing higher concentrations of both sugar and inhibitors. In FIG. 9, the resulting increasing ethanol over the 70 day fermentation can be observed in the upper panel. Sugar concentrations higher than 123 g/L were not tried. Two aspects that affect the fermentation performance are ethanol and other inhibitor concentrations.

Several fermentations have tested ethanol tolerances and have all failed at concentrations ranging between 30 and 35 g/L within the fermenter using non-toxic influents.

It has also been shown that the presence of inhibitors decrease the tolerance to ethanol.

6.9% ethanol is an average value covering a period of 25 days (FIG. 9, upper panel).

### Example 2

### Continuous Thermophilic Fermentation on Laboratory Scale Using N₂ Stripping (Enzymatically Hydrolyzed Pretreated Wheat Straw)

The continuous reactor system was used to test fermentation of pretreated and enzyme hydrolyzed wheat straw in connection with nitrogen sparging. Two fermentation strategies were used. In a first, real hydrolysates were used and no additional sugar was added, as the enzymatic hydrolysis had released the glucose from cellulose (Table 1, B). The highest fermentable dry matter concentration was 19.2% DM (before separation and corrected for base titration). Ethanol concentrations exceeded 49 g/L (6.1% vol/vol). The second fermentation was performed on the liquid fraction from pretreated biomass with added glucose to simulate enzymatically hydrolyzed pretreated wheat straw (Table 1, C). Carbohydrate feed concentrations are shown in Table 1 below. These later data showed ethanol concentrations of 60 g/L (7.5% vol/vol) in the steady state of the fermentation. At this ethanol concentration, sugar conversion was still high indicating that the fermentation was limited by the availability of sugar rather than toxicity.

### Example 3

### Continuous Thermophilic Fermentation on Laboratory Scale Using N₂ Stripping

A similar setup as described above, using a glass reactor with a smaller active volume (445 mL) was used testing co-fermentation in combination with nitrogen sparging. The fermentation was started at batch conditions and glucose-enriched C5 liquor media were gradually increased in sugar concentration throughout the experiment. The influent with the highest sugar concentration originated from pretreated wheat straw with a dry matter concentration of 15% and contained around 63 g/L and 41 g/L of glucose and xylose, respectively. During steady state, ethanol concentrations reached 46 g/L (5.8% vol/vol) and the feed rate was gradually increased to 4.5 g/L/h (Table 1, D). The inhibitory compounds, originally present in the pretreated wheat straw, were undetectable in both broths taken directly from the fermenter and in condensed effluents. The sugar conversion remained high during the entire experiment at approximately 99%.

Nitrogen sparging (Table 1, B,C,D) was found to be comparable to CO₂ sparging (A).

TABLE 1
A Summary of different thermophile fermentations on either C5 and C6- or only C5 [symbol]
gars. The table shows fermentations according to Examples 1, 2 and 3 on influents containing [symbol]
ther lignocellulose derived C5 sugar only (F and G), lignocellulosic C5 sugars with added [symbol]
nthetic glucose (A, C, D, and E) or lignocellulose derived C5 and C6 sugars (lignocellulosic [symbol]
drolysate produced using enzymes) (B) using either suspended cell continuous stirred tank [symbol]
stems (A, B, C, D, and E) or using immobilized microorganisms (F and G).

| | | | | | | | | | HMF | | |
|---|---|---|---|---|---|---|---|---|---|---|---|
| | Ethanol | Ethanol | Carbohydrate | Ethanol | | Sugars | Furfural | Acetate | in | Sugar | No. |
| | Conc¹ | productivity | feed rate² | yield³ | DM⁴ | in feed⁵ | in feed⁶ | in feed | feed⁶ | conversion | of |
| | g/L | g/L/h | g/L/h | g/g | % | g/L | g/L | g/L | g/L | % | days |
| CSTR with | 58 | 1.7 | 3.5 | 0.48 | 17.9 | 123 | 0.7 | 5.7 | 0.1 | 100 | 25 |
| ethanol | | | | | | | | | | | |
| removal | | | | | | | | | | | |
| CO₂ | | | | | | | | | | | |
| CSTR with | 54 | 1.7 | 3.5 | 0.49 | 19.2 | 106 | 0.4 | 6.0 | 0.1 | 100 | 23 |
| ethanol | | | | | | | | | | | |
| removal | | | | | | | | | | | |
| N₂, | | | | | | | | | | | |
| Hydrolysate | | | | | | | | | | | |
| CSTR with | 60 | 1.7 | 3.5 | 0.49 | 17.8 | 123 | 0.7 | 6.0 | 0.1 | 100 | 22 |
| ethanol | | | | | | | | | | | |
| removal | | | | | | | | | | | |
| N₂ | | | | | | | | | | | |
| CSTR with | 46 | 2.0 | 4.5 | 0.47 | 15 | 100/103 | 0.41 | 4.4 | 0 | 99 | 49 |
| ethanol | | | | | | | | | | | |
| removal | | | | | | | | | | | |
| N₂ | | | | | | | | | | | |
| CSTR without | 38 | 0.9 | 1.9 | 0.48 | 9.2 | 81 | 0.4 | 3.4 | 0.05 | 100 | 6 |
| ethanol | | | | | | | | | | | |
| removal | | | | | | | | | | | |
| Immobilization | 9 | NA | NA | 0.40 | 10 | 25 | NA | NA | NA | 89 | NA |
| without | | | | | | | | | | | |
| removal | | | | | | | | | | | |
| Ref: | | | | | | | | | | | |
| Georgieva | | | | | | | | | | | |
| (2007) | | | | | | | | | | | |
| Immobilization | 9 | NA | NA | 0.35 | 15 | 40 | NA | NA | NA | 70 | NA |
| without | | | | | | | | | | | |
| removal | | | | | | | | | | | |
| Ref: WO | | | | | | | | | | | |
| 2007/1341607 | | | | | | | | | | | |
| A1 | | | | | | | | | | | |

[symbol]

orrected values based carbon recoveries,
²Gram carbohydrates per liter feed per hour,
³Gram [symbol] anol produced per gram sugar conversed,
⁴Dry matter concentration after pre-treatment and [symbol] fore separation.
⁵Corrected concentrations including base titration,
⁶Furfural and HMF are [symbol] detectable in the reactor. [symbol]
indicates data missing or illegible when filed

Fermentation of lignocellulosic biomass into ethanol is typically done using either yeast in batch processes or bacteria in either batch or continuous processes. Continuous fermentation systems have advantages over batch fermentations as they allow the microorganisms to adapt to the inhibitors present in the biomass, and they enable higher productivities and yields. However, these systems are more challenging to operate due to the risk of contamination. Fermentation using thermophilic microorganisms allows long term operation because of the high operating temperature. However, thermophilic microorganisms have not previously been demonstrated to be able to grow in highly concentrated lignocellulosic biomass in continuous, fully suspended fermentation reactors such as stirred tank reactors, since they were generally not believed to be able to sustain a high growth rate if cell retention systems such as immobilization were not employed (WO2007134607). As can be seen from Table 1, a standard continuous stirred tank bioreactor, without means of partially removing ethanol, could not be operated at biomass drymatter concentration above 9.2% (Table 1, E).

Above that an increase in unfermented sugars was seen and sugar conversion as measured by HPLC of reactor samples relative to influent samples gradually decreased, despite prolonging the hydraulic retention time. If immobilization was employed, drymatter concentrations up to 15% were possible in immobilized cell reactor systems (Table 1, F and G). The results in Table 1 (A, B and C) show that using partial ethanol removal, drymatter concentrations of up to 19.5% can be achieved using thermophilic bacteria with a resulting ethanol concentration of at least 54 g/L in the fermentation effluent (including recovered ethanol from the ethanol removal system). In addition, high ethanol yields and ethanol productivities were achieved using the present process.

Furfural arising from degradation of xylose released in pre-treatment was completely converted in the continuous fermentations as no furfural was detected in the samples from the fermentation reactor or the from fermentation effluent (data not shown). Since furfural is a known inhibitor of fermentation processes, this demonstrates a significant advantage of using the process of the present invention. The continuous operation at high pre-treated biomass drymatter concentrations is made possible by the use of thermophilic bacteria which continuously consume fermentation inhibitors. Surprisingly, the partial removal of ethanol resulted in growth rates sufficiently high to sustain a high productivity in fully suspended continuous stirred tank reactors, even if the biomass had not otherwise been detoxified, and industrially relevant fermentation with thermophilic bacteria is thereby made possible.

### Example 4

### Vacuum Fermentation Model

The function of continuous removal of ethanol from an ongoing fermentation using vacuum (FIG. 3) was simulated by ChemCAD 6.4.3, in order to verify that suitable growth conditions for the bacteria could be maintained.

In the ChemCAD modeling, the setup consists of a main tank (FIG. 3, 1) and a smaller vacuum tank (FIG. 3, 10. Total working volume 100 m³). The fermentation broth is recycled between the two tanks. The overall flow rates are presented in Table 2. The modeled fermentation broth includes the major ions as 50₄²⁻, HSO₄⁻, K⁺, NH₄⁺, HPO₄²⁻, H₂PO₄⁻, HCO₃⁻ and Cl⁻ (from the feedstock) giving a ionic strength of 0.281 mol/kg in the feed (initial pH 1.2) as well as gasses (CO₂) and vapors (H₂O and ethanol). The model explores the ethanol removal only and thus instead of a sugar feed entering the fermentor a theoretical ethanol concentration of 55.2 g/L was used. Compared to real fermentation this corresponds to typical values: sugar 120 g/L, yield 0.47 g/g and conversion 98%. The hydraulic retention time was fixed at 30 h corresponding to a productivity of 1.84 g/L/h or 184 kg/h for the modeled fermentor system. The corresponding CO₂ production rate used was 176 kg/h.

The ChemCAD model runs to a steady state situation with a stable ethanol concentration below the chosen maximum of 31 g/L in the fermentor. In Table 2, results of the model can be seen.

TABLE 2
Results from ChemCAD modeling of fermentation with
vacuum ethanol removal. The number connected to the different
flows refers to FIG. 3, (in the model the effluent liquid
is removed from the vacuum tank). The feed rate is 3.36 T/h
(including 0.02 T/h for pH adjustment) and the recirculation
rate 113 T/h. The liquid effluent is 2.69 T/h and the vapor
flow is 0.67 T/h.

| | | | Vacuum | |
|---|---|---|---|---|
| | | | tank/ | |
| | | | effluent | |
| | | | liquid | |
| | | Fermentor | (10, 9 | Effluent |
| Parameter | “Feed” (3) | (2) | (4)) | vapor (9) |
| Temperature | 66 | 68.2 | 68.0 | 68.0 |
| (° C.) | | | | |
| Pressure | 1 | 1.0 | 0.36 | 0.36 |
| (bara) | | | | |
| pH | 1.2 | 6.4 | 7.5 | — |
| Ionic str. | 0.281 | 0.508 | 0.515 | — |
| (molal) | | | | |
| Ethanol | 5.52 | 3.08 | 3.01 | 15.4 |
| (w/w %) | | | | |

During exposure to the low pressure in the vacuum zone, the recirculating fermentation broth gently boils creating an effluent vapor flow of water, ethanol and CO₂. The remaining liquid phase, which returns to the fermentor (or part of it leaves the system as liquid effluent) thus has lower ethanol concentration, higher salt concentrations and higher pH. A high energy input to the vacuum tank keeps the temperature close to the situation in the main tank. The energy part of the model, which is integrated with the condensation/distillation part of the model is not shown. The parameters revealed by the ChemCAD evaluation (Table 2) are not in the inhibitory range observed for ethanol production by the Pentocrobe™ (Larsen L et al. 1997). The impact of high or low pressure on bacterial growth was investigated in a separate laboratory experiment. In batch, initial pressure of 2.0 bara or 0.4 bara did not inhibit initiation of growth). Changes in the production due to e.g changes in initial sugar concentration can to a large extent be adjusted for by changing the parameters included in the model such as production rate, recirculation rate etc. An overall limit here is the removal of water with the ethanol vapor. In this respect, the vacuum fermentation is expected to behave as the N₂ sparged fermentations described in Example 1 above.

Thus, the ChemCAD simulation strongly supports that using vacuum ethanol removal, it is possible to keep the ethanol concentration below inhibitory levels without compromising the overall growth conditions.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### LIST OF REFERENCES

Amartey S. A., Leung P. C. J., Baghaei-Yazdi N., Leak D. J., Hartley B. S., Fermentation of a wheat straw acid hydrolysates by Bacillus stearothermophilus T-13 in continuous culture with partial cell recycle, Process Biochemicstry 34 (1999) 289-294.

Hemme, C. L., M. W. Fields, et al. (2011). Correlation of genomic and physiological traits of thermoanaerobacter species with biofuel yields. Appl Environ Microbiol 77(22): 7998-8008.

Kumar, S., S. P. Singh, Taylor et al. (2009). Recent Advances in Production of Bioethanol from Lignocellulosic Biomass. Chemical Engineering & Technology 32(4): 517-526.

Najafpour, G. D. 2007. Biochemical Engineering and Biotechnology. Elsevier.

Taylor et al., 2010, Continuous high-solids corn liquefaction

Hungate, R. E. (1969). A roll tube method for cultivation of strict anaerobes. Methods in microbiology. J. R. Norris and D. W. Ribbons. New York, Academic Press: 118-132.

Larsen, L., P. Nielsen, B. K. Ahring. (1997). “Thermoanaerobacter mathranii sp. nov., an ethanol producing, extremely thermophilic anaerobic bacterium from a hot spring in Iceland.” Arch. Microbiol. 168(2): 114-119.

S., Wyman, C. E. (2010). Review: Continuous hydrolysis and fermentation for cellulosic ethanol production. Bioresource Technology 101, 4862-4874.

1. A method for the production of ethanol comprising feeding a fermentable lignocellulosic biomass feed into a continuous fermentation, said fermentable lignocellulosic biomass feed having being obtained by treatment of a starting lignocellulosic biomass to liberate carbohydrates contained therein and containing the carbohydrates and associated fermentation inhibiting biomass components including at least one of hydroxymethylfurfural, 2-furaldehyde and acetic acid produced from said starting biomass together with the carbohydrates in said treatment, and continuously fermenting fermentable carbohydrate components of said biomass feed at an elevated temperature using an obligatorily anaerobic thermophilic microorganism which is suspended and not immobilised, wherein ethanol is continuously or continually removed during the fermentation and wherein said feed contains a concentration of the fermentation inhibiting compound hydroxymethylfurfural of at least 0.05 g/L, or contains a concentration of the fermentation inhibiting compound 2-furaldehyde of at least 0.5 g/L, or a concentration of the fermentation inhibiting compound acetic acid of at least 5 g/L.

2. A method as claimed in claim 1, further comprising a preceding step of conducting said treatment of said starting lignocellulosic biomass to provide said fermentable lignocellulosic biomass feed.

3. A method as claimed in claim 2, wherein said treatment comprises pre-treating said lignocellulosic biomass to liberate crude C5 monosaccharides and to liberate crude polysaccharides for hydrolysis.

4. A method as claimed in claim 3, further comprising hydrolysing said polysaccharides to provide crude C6 monosaccharides in said feed.

5. A method as claimed in claim 4, wherein hydrolysis of said crude polysaccharides is conducted by enzymes added to the pre-treated lignocellulosic biomass.

6. A method as claimed in claim 3, wherein hydrolysis of said polysaccharides is conducted by enzymes added to said fermentation.

7. A method as claimed in claim 1, wherein ethanol is removed from the fermentation by gas stripping using a stripping gas.

8. A method as claimed in claim 7, wherein ethanol is removed from admixture with the stripping gas and thus purified stripping gas is reused for ethanol removal.

9. A method as claimed in claim 1, wherein ethanol is removed from the fermentation by the use of vacuum.

10. A method as claimed in claim 1, wherein said removal of ethanol is conducted on a liquid stream withdrawn from the fermentation into a separate vessel from a vessel in which said fermentation is carried out.

11. A method as claimed in claim 1, wherein the microorganism is a filamentous microorganism.

12. A method as claimed in claim 1, wherein the microorganism is from the class of Clostridia.

13. A method as claimed in claim 1, wherein the microorganism is from the order of Thermoanaerobacteriales.

14. A method as claimed in claim 1, wherein the microorganism is from the family of Thermoanaerobacteriaceae.

15. A method as claimed in claim 1, wherein the microorganism is from the genus of Thermoanaerobacter.

16. A method according to claim 1, wherein the microorganism is selected from the group consisting of Thermoanaerobacter acetoethylicus, Thermoanaerobacter brockii, Thermoanaerobacter ethanolicus, Thermoanaerobacter inferii, Thermoanaerobacter italicus, Thermoanaerobacter italicus subsp. marato, Thermoanaerobacter keratinophilus, Thermoanaerobacter kivui, Thermoanaerobacter mathranii, Thermoanaerobacter pseudethanolicus, Thermoanaerobacter siderophilus, Thermoanaerobacter sulfurigignens, Thermoanaerobacter sulfurophilus, Thermoanaerobacter thermocopriae, Thermoanaerobacter thermohydrosulfuricus, Thermoanaerobacter uzonensis and Thermoanaerobacter wiegelii.

17. A method as claimed in any preceding claim 1, wherein the carbohydrate content of said feed is at least 125 g/L.

18. A method as claimed in claim 1, wherein the carbohydrate content of said feed is at least 150 g/L.

19. The method according to claim 1, wherein a portion of the fermentation broth is removed during the continuous fermentation process, and the microorganisms are recycled back into the fermentation vessel.

20. The method according to claim 19, wherein the microorganisms are recycled by:
isolating a portion of the fermentation broth, isolating microorganisms from said portion of fermentation broth,
optionally, treating said isolated microorganisms, and
re-introducing said isolated microorganisms into the fermentation broth.

21. The method according to claim 20, wherein said isolated microorganisms are treated using a treatment selected from the group consisting of heat treatment, acid or base treatment with or without increased pressure, and enzymatic lysis.

22. The method according to claim 20, wherein isolating of the microorganisms is performed by continuous centrifugation.

23. The method according to claim 20, wherein isolation of the microorganisms takes place via filtration.

24. The method according to claim 1, wherein the rate of carbohydrate feed to the fermentation is at least 0.5 g per litre of fermentation volume per hour.

25. The method according to claim 1, wherein the fermentable lignocellulosic biomass feed contains at least 80% of at least one associated fermentation inhibiting biomass component produced from said starting biomass together with the carbohydrates in said treatment.

26. The method according to claim 1, wherein the fermentable lignocellulosic biomass feed contains all of the associated fermentation inhibiting biomass components produced from said starting biomass together with the carbohydrates in said treatment.

27. The method according to claim 1, wherein lignin is removed from the biomass following said treatment and prior to feeding to said fermentation.

28. The method according to claim 4, wherein the fermentable lignocellulosic biomass feed contains both the C5 and C6 monosaccharides liberated in said treatment.

29. The method according to claim 1, wherein said feed contains at least two of:
a concentration of the fermentation inhibiting compound hydroxymethylfurfural of at least 0.05 g/L,
a concentration of the fermentation inhibiting compound 2-furaldehyde of at least 0.5 g/L, and
a concentration of the fermentation inhibiting compound acetic acid of at least 5 g/L.

30. The method according to claim 1, wherein said feed contains all of:
a concentration of the fermentation inhibiting compound hydroxymethylfurfural of at least 0.05 g/L,
a concentration of the fermentation inhibiting compound 2-furaldehyde of at least 0.5 g/L, and
a concentration of the fermentation inhibiting compound acetic acid of at least 5 g/L.

31. The method according to claim 1, wherein said elevated temperature is at least 50° C.

32. The method according to claim 1, wherein the concentration of ethanol in the fermentation medium is kept below 45 g/L.

33. The method according to claim 1, wherein said ethanol removal removes from the fermentation medium at least 10% of the ethanol produced in said fermentation.
